(19) United States
(12) Patent Application Publication
(10) Pub. No.: US 2022/0400990 A1
(43) Pub. Date: Dec. 22, 2022

(54) EAR WEARING TYPE SENSOR PROBE FOR MEASURING SPO2

(71) Applicant: BIONET CO., LTD, Seoul (KR)

(72) Inventor: Ki Young LEE, Seoul (KR)

(73) Assignee: BIONET CO., LTD, Seoul (KR)

(21) Appl. No.: 17/839,705

(22) Filed: Jun. 14, 2022

(30) Foreign Application Priority Data
Jun. 17, 2021 (KR) 10-2021-0078417

(51) Int. Cl. A61B 5/1455; A61B 5/145; A61B 5/00

(52) U.S. Cl. CPC A61B 5/14552; A61B 5/14546; A61B 5/6817

(57) ABSTRACT

Provided is an ear wearing type sensor probe for measuring oxygen saturation (SpO2). The ear wearing type sensor probe is configured such that the oxygen saturation (SpO2) may be measured while worn in an outer ear canal with abundant blood vessels and close to the heart in the form of an earphone, so that the user's hand may move freely and motion artifact noise may be minimized to measure relatively accurate oxygen saturation (SpO2).

### CROSS REFERENCE

The present application claims priority to and the benefit of Republic of Korea patent application no. 10-2021-0078417 filed Jun. 17, 2021.

### BACKGROUND OF THE DISCLOSURE

### Field of the Disclosure

The present disclosure relates to an ear wearing type sensor probe for measuring an oxygen saturation, and more specifically, to an ear wearing type sensor probe for measuring an oxygen saturation (SpO2), which is configured such that the oxygen saturation (SpO2) may be measured while worn in an outer ear canal with abundant blood vessels and close to the heart in the form of an earphone, so that the user's hand may move freely and motion artifact noise may be minimized to measure relatively accurate oxygen saturation (SpO2).

### Related Art

In general, when measuring oxygen saturation (SpO2), a sensor is configured to be coupled to the patient's finger to measure an oxygen saturation of arterial blood by checking a blood flow condition of the finger to measure the oxygen saturation, and then displaying it on the display. Specifically, it is configured to continuously measure an oxygen saturation of arterial blood in a non-invasive manner using properties that an amount of light sensed by the dilation and contraction of pulsatile arterial blood vessels is influenced by a sensitive fraction between unsaturated hemoglobin and saturated hemoglobin.

A detachable pulse oximeter as described above is disclosed in Korean Patent Registration No. 10-0756654 (hereinafter referred to as “Patent Document 1”).

Referring to the Patent Document FIG. 1, the conventional oximeter comprises a first housing 1 including a first upper body 11, a first lower body 12 coupled to the lower portion of the first upper body 11, and a circuit board 13 coupled between the first upper body 11 and the first lower body 12; a second housing 2 including a second upper body 21 and a second lower body 22 coupled to a lower portion of the second upper body 21; and a third housing 3 including a third upper body 31 and a third lower body 32 coupled to the lower portion of the third upper body 31 and accommodating a battery 7 therein; wherein the first housing 1 and the second housing 2 are detachably coupled by a coupling means 5, a tab 212 formed in the second housing is pivotally coupled to a tab coupling portion 321 formed in the third housing, and the second housing 2 and the third housing 3 are configured such that a spring 6 having a bent portion 61 and an coupling portion 62 is coupled to a spring coupling portion 224 of the second housing and a spring fixing portion 311 of the third housing.

In the Patent Document 1, the oximeter vice having the above configuration is configured such that a body for measuring the oxygen saturation of arterial blood by sensing the light transmitted through the finger and including a display unit displaying the measured value may be detachable and coupled to a measurement unit. However, as described above, the finger-type oximeter has limitations in the user's hand movement, and artifact corruption occurs due to motion artifacts caused by the user's hand movement, thereby affecting a photo-plethysmography measurement signal (PPG). For this reason, there was a problem in that the output of the pulse oximeter was greatly changed, resulting in a large difference in the value of the oxygen saturation (SpO2).

### SUMMARY OF THE DISCLOSURE

The present disclosure has been devised to solve the above problems. Accordingly, an object of the present disclosure is to provide an ear wearing type sensor probe for measuring oxygen saturation (SpO2), which is configured such that the oxygen saturation (SpO2) may be measured while worn in an outer ear canal with abundant blood vessels and close to the heart in the form of an earphone, so that the user's hand may move freely and motion artifact noise may be minimized to measure relatively accurate oxygen saturation (SpO2).

In order to solve the above problems, according to a preferred embodiment of the present disclosure, an ear wearing type sensor probe for measuring oxygen saturation may include a probe body, and the probe body may include an infrared LED for measuring the concentration of hemoglobin in oxygenated blood, a red LED for measuring the concentration of hemoglobin in blood from which oxygen has been removed, and a sensor board including a photodetector for detecting the reflected light reflected from the blood by the infrared LED and the red LED and converting it into an electrical signal, wherein it may be configured to measure the oxygen saturation (SpO2) in a portion of the outer ear canal of the ear while the probe body is worn on the user's ear in the form of an earphone.

In addition, the probe body may include an insertion part inserted into the user's ear and being in close contact with the outer ear canal, and a seating part positioned at the distal end of the insertion part to be seated on the user's ear.

In addition, the probe body may have a hearing hole penetrating through the insertion part from the seating part so that the user may listen while worn on the user's ear.

In addition, the outer peripheral surface of the insertion part may be provided with a coating part treated with a transparent epoxy coating to provide light transmittance and adhesion in the process of being in contact with the user's ear.

In addition, the insertion part may be configured such that the oxygen saturation (SpO2) is measured while the ear wearing type sensor probe is worn in a range between 8 and 12 mm in the ear, which corresponds to the outer ⅓ of the entire length of the outer ear canal.

As described above, according to the present disclosure, there is an advantage in that it is configured such that the oxygen saturation (SpO2) may be measured while worn in an outer ear canal with abundant blood vessels and close to the heart in the form of an earphone, so that the user's hand may move freely and motion artifact noise may be minimized to measure relatively accurate oxygen saturation (SpO2).

### BRIEF DESCRIPTION OF THE DRAWINGS

FIG. 1 is a diagram showing a configuration of an ear wearing type sensor probe for measuring an oxygen saturation (SpO2) according to a preferred embodiment of the present disclosure;

FIG. 2 is a block diagram showing a configuration of a sensor board of the ear wearing type sensor probe for measuring the oxygen saturation according to a preferred embodiment of the present disclosure;

FIG. 3 is an explanatory view showing a wearing position of an ear wearing type sensor probe for measuring the oxygen saturation according to a preferred embodiment of the present disclosure; and

FIG. 4 is an explanatory view showing a state in which the ear wearing type sensor probe for measuring the oxygen saturation is worn on an ear according to a preferred embodiment of the present disclosure.

### DESCRIPTION OF EXEMPLARY EMBODIMENTS

Hereinafter, the present disclosure will be described in detail with reference to the drawings. Like reference numerals proposed in each drawing denote like elements.

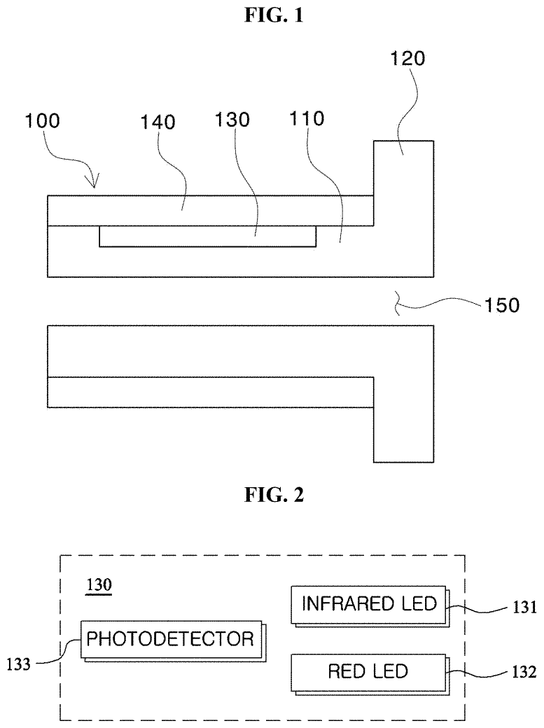

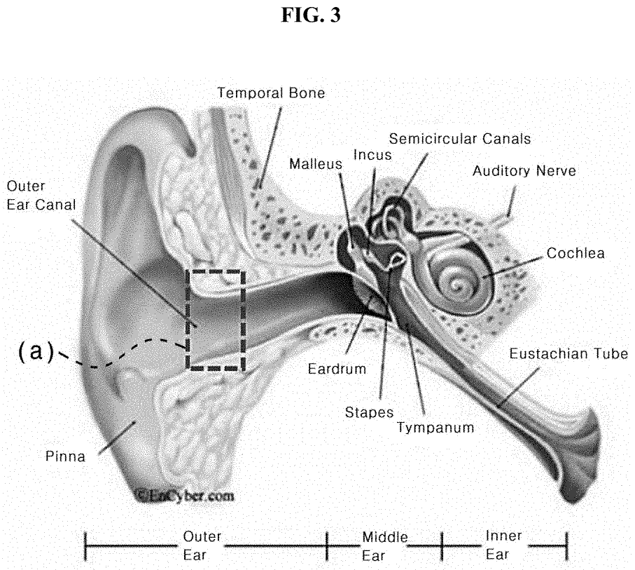

FIG. 1 is a diagram showing a configuration of an ear wearing type sensor probe for measuring an oxygen saturation (SpO2) according to a preferred embodiment of the present disclosure, and FIG. 2 is a block diagram showing a configuration of a sensor board of the ear wearing type sensor probe for measuring the oxygen saturation according to a preferred embodiment of the present disclosure.

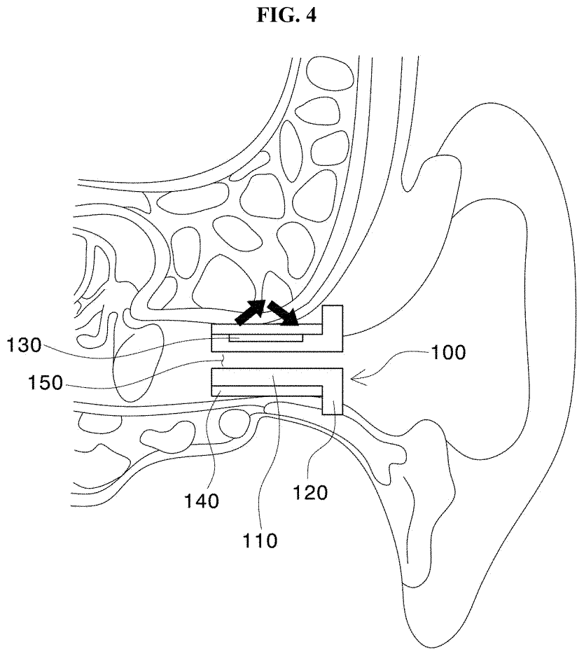

FIG. 3 is an explanatory view showing a wearing position of an ear wearing type sensor probe for measuring the oxygen saturation according to a preferred embodiment of the present disclosure, and FIG. 4 is an explanatory view showing a state in which the ear wearing type sensor probe for measuring the oxygen saturation is worn on an ear according to a preferred embodiment of the present disclosure.

Referring to FIGS. 1 to 4, according to a preferred embodiment of the present disclosure, the ear wearing type sensor probe for measuring the oxygen saturation is configured such that the oxygen saturation (SpO2) may be measured in a portion of the outer ear canal while worn in the user's ear in the form of an earphone. To this end, the ear wearing type sensor probe may include a probe body 100 including an insertion part 110 inserted into the user's ear and being in close contact with the outer ear canal, and a seating part 120 positioned at the distal end of the insertion part 110 to be seated on the user's ear.

Usually, since a large number of blood vessels are distributed in the outer ear canal, and the outer ear canal is closer to the heart than measuring the oxygen saturation (SpO2) by connecting a forceps-shaped measuring part to a finger, it is possible to measure the oxygen saturation (SpO2) efficiently. In the present disclosure, the ear wearing type sensor probe may be configured such that the oxygen saturation (SpO2) may be measured while wearing the probe body 100 at a point (a) between 8 and 12 mm in the ear, which corresponds to the outer ⅓ of about 25 to 35 mm, which is the entire length of the outer ear canal. Accordingly, in addition to free movement of the user's hand, it is possible to measure relatively accurate oxygen saturation (SpO2) by minimizing motion artifact noise.

In addition, a point that enters the inner ear canal through the outer ⅓ of the outer ear canal is vulnerable to infections such as bacteria. However, according to the present disclosure, it is possible to safely measure the oxygen saturation (SpO2) at a point corresponding to the outer ⅓ of the outer ear canal, that is, at the maximum outer side of the ear.

In addition, it is preferable that the probe body 100 may be configured to be used in connection with a pulse-oximeter, and to be disposable to prevent cross-infection.

Meanwhile, the inner side of the probe body 100 may be provided with an infrared LED 131 for measuring the concentration of hemoglobin in oxygenated blood, a red LED 132 for measuring the concentration of hemoglobin in blood from which oxygen has been removed, and a sensor board 130 including a photodetector 133 for detecting the reflected light reflected from the blood by the infrared LED 131 and the red LED 132 and converting it into an electrical signal. As shown in FIG. 4, the ear wearing type sensor probe may be configured to measure the oxygen saturation (SpO2) through a blood vessel surface reflection type sensing method while worn on the outer ear canal of the user's ear.

In addition, it is preferable that the probe body 100 is provided with a hearing hole 150 penetrating through the insertion part 110 from the seating part 120 so that the user may listen while wearing the probe in the user's ear.

In addition, it is preferable that the outer peripheral surface of the insertion part 110 is provided with a coating part 140 treated with a transparent epoxy coating to provide light transmittance and adhesion in the process of being in contact with the user's ear.

As described above, the present disclosure may be configured such that the oxygen saturation (SpO2) may be easily measured while worn in an outer ear canal with abundant blood vessels and close to the heart in the form of an earphone, so that the user's hand may move freely and motion artifact noise may be minimized to measure relatively accurate oxygen saturation (SpO2).

Best embodiments have been disclosed in the drawings and specification. Although specific terms are used herein, they are used only for the purpose of describing the present disclosure, and are not used to limit the meaning or scope of the present disclosure described in the claims. Accordingly, those skilled in the art will understand that various modifications and equivalent other embodiments are possible therefrom. Therefore, the true technical protection scope of the present disclosure should be determined by the technical spirit of the appended claims.

What is claimed is:

1. An ear wearing type sensor probe for measuring oxygen saturation (SpO2), which including a probe body, the probe body comprising:
an infrared LED for measuring the concentration of hemoglobin in oxygenated blood;
a red LED for measuring the concentration of hemoglobin in blood from which oxygen has been removed; and
a sensor board including a photodetector for detecting the reflected light reflected from the blood by the infrared LED and the red LED and converting it into an electrical signal,
wherein it is configured to measure the oxygen saturation (SpO2) in a portion of the outer ear canal of the ear while the probe body is worn on the user's ear in the form of an earphone.

2. The ear wearing type sensor probe of claim 1, wherein the probe body includes an insertion part inserted into the user's ear and being in close contact with the outer ear canal, and a seating part positioned at the distal end of the insertion part to be seated on the user's ear.

3. The ear wearing type sensor probe of claim 2, wherein the probe body has a hearing hole penetrating through the insertion part from the seating part so that the user may listen while worn on the user's ear.

4. The ear wearing type sensor probe of claim 2, wherein the outer peripheral surface of the insertion part is provided with a coating part treated with a transparent epoxy coating to provide light transmittance and adhesion in the process of being in contact with the user's ear.

5. The ear wearing type sensor probe of claim 2, wherein the insertion part is configured such that the oxygen saturation (SpO2) is measured while the ear wearing type sensor probe is worn in a range between 8 and 12 mm in the ear, which corresponds to the outer ⅓ of the entire length of the outer ear canal.
